(19) United States
(12) Patent Application Publication
(10) Pub. No.: US 2012/0123293 A1
(43) Pub. Date: May 17, 2012

(54) MOTOR NERVE ROOT STIMULATION

(75) Inventors: Jawad A. Shah, Flushing, MI (US); Amer Iqbal, Flint, MI (US); Richard H. Smith, Dexter, MI (US)

(73) Assignee: IINN, Inc., Flint, MI (US)

(21) Appl. No.: 13/293,964

(22) Filed: Nov. 10, 2011

Related U.S. Application Data
(60) Provisional application No. 61/412,484, filed on Nov. 11, 2010.

(51) Int. Cl. A61N 1/36; A61B 5/11; A61H 3/00; A61M 37/00

(52) U.S. Cl. 600/554; 607/48; 607/49; 607/46; 607/3

(57) ABSTRACT

A motor device for bypassing or bridging an area of neurological damage, including at least one electrode having an electric mechanism for generating electric current, and a programming mechanism for programming the electrode. A biofeedback system and an artificial spinal cord. A method of stimulating muscle in a person having neurological damage, by applying electric current to nerves, bypassing or bridging an area of neurological damage, and moving the muscle in a natural manner. Methods of moving muscles of a paraplegic, reducing or eliminating pain from an individual, and treating foot drop. A therapeutic method for exercising for an individual with neurological damage. A method of generating movement of muscle and sensing that movement in a person having neurological damage. A method of generating movement of muscle using information from the brain and sensing that movement in a person having neurological damage. A method of diagnosing neurological damage.

### BACKGROUND OF THE INVENTION

1. Technical Field

The present invention relates to electrical stimulation of nerves. In particular, the present invention relates to electrical stimulation of nerves to move muscles and regain function of the body after neurological damage.

2. Background Art

Circumstances arising from trauma, such as motor vehicle accidents, falls, etc., can result in neurological damage involving the spinal cord such that the individual becomes paralyzed or loses feeling in one or many parts of the body. In addition, there are a variety of diseases that involve the spinal cord, and can cause progressive loss of muscular control, severely reducing the normal function and quality of life of the individual. Likewise, there are a variety of conditions that can cause an individual to feel pain, whether chronically or episodically, in various parts of the body. These and other causes and conditions present significant challenges to individuals and their families who strive to help the afflicted person cope with such disabilities.

The spinal cord extends from the base of the brain to about the waist, extending to the space between the first and second lumbar vertebrae, and is protected by bony processes in the vertebral column. The eight vertebrae in the neck are called cervical vertebrae, and the one at the top is labeled C-1. Following the cervical vertebrae are the twelve thoracic, five lumbar, and five sacral vertebrae.

The spinal cord nerves, known as the upper motor neurons, function to carry electrical impulses to and from the brain to spinal nerves along the spinal tract. Lower motor neurons are the spinal nerves that branch out from the spinal cord and communicate with specific regions in the body, initiating actions such as muscle movement. The lower motor neurons emanate from specific vertebrae such that injury to the spinal cord at a particular vertebra causes specific dysfunction in the afflicted person. In general, injuries higher up in the vertebral column will cause higher levels of dysfunction. The central problem with neurological damage in the spinal column is the loss of communication along nerves at various levels of the spinal column.

There are several methods that are currently used to treat spinal column damage. Surgery, a variety of drugs, and physical therapy are currently used. Experimental treatments include use of stem cell, autologous transplants, and genetically engineered biological agents.

For example, methylprednisolone is often given within eight hours of injury, and while not a cure, it has shown to provide mild improvement through reducing damage to nerve cells and decreasing inflammation; however, this treatment has fallen out of favor due to complications. Surgery can be used to repair disks or vertebrae that are compressing the spine. Physical therapy is used to help persons relearn how to move muscles or strengthen other muscles needed to perform tasks that were previously done with other muscles.

None of these treatments has a high success rate with paraplegics. Surgery has attendant risks, and frequently results in fibrosis at the surgical site. The use of drugs may not effectively target specific tissues, because drugs must pass through general circulation to get to the afflicted site, and there may be many adverse effects, such as liver damage and other unintended consequences of treatment. Physical therapy seems to provide mostly palliative results, due to increased blood flow in exercising of limbs, and cannot provide neurological stimulation at specific sites when it is needed. Experimental treatments may only be available in clinical studies, only in specified institutions or locations, and so on, and they often have not been fully tested for safety and efficacy. Complete spinal disruption, anatomical or physiological, has no current treatment for the complete return of function.

Spinal cord stimulators have been used to reduce chronic pain by implantation of wires near the spinal cord. The reduction rate can be 50% or greater. Chronic pain is reduced by interrupting nerve conduction of the pain with low level electrical stimulation produced by a spinal cord stimulator. In essence, the spinal cord stimulator produces an electrical current that competes for the brain's attention with the pain, such that the brain focuses on the electrical current and not the pain.

U.S. Pat. No. 7,610,096 to McDonald, III, discloses methods for the treatment of CNS damage, and includes inducing in a subject in need of such treatment, a therapeutically effective amount of functional electrical stimulation (FES) sufficient to evoke patterned movement in the subject's muscles, the control of which has been affected by the CNS damage. The induction of FES-evoked patterned movement at least partially restores lost motor and sensory function, and stimulates regeneration of neural progenitor cells in the subject person. The treatment is thought to work by inducing FES-evoked patterned body movements that regenerate neural cells such that CNS damage previously thought beyond repair is repaired, and function previously thought permanently lost is at least partially restored. Without being bound to a particular theory, the FES-evoked patterned movements are thought to stimulate neural regeneration by stimulating neural activity in a central pattern generator. Physiologic and metabolic demands placed on cells comprising the spinal circuit may activate cellular processes that promote new neural cell birth and survival. FES can thereby harness the innate plasticity of the nervous system. While recovery of function is possible to the extent that neurons can be created, this particular method does not provide a way to recover function when the repair or regeneration needed is too great or not possible.

U.S. Pat. No. 7,778,704 to Rezai discloses a method of affecting physiological disorders by stimulating a specific location along the sympathetic nerve chain. A method is disclosed of affecting a variety of physiological disorders or pathological conditions by placing an electrode adjacent to or in communication with at least one ganglion along the sympathetic nerve chain and stimulating the at least one ganglion until the physiological disorder or pathological condition has been affected. Physiological disorders that may be treated include, but are not limited to, hyperhydrosis, complex regional pain syndrome and other pain syndromes such as headaches, cluster headaches, abnormal cardiac sympathetic output, cardiac contractility, excessive blushing condition, hypertension, renal disease, heart failure, angina, hypertension, and intestinal motility disorders, dry eye or mouth disorders, sexual dysfunction, asthma, liver disorders, pancreas disorders, and heart disorders, pulmonary disorders, gastrointestinal disorders, and biliary disorders.

Harkema, et al. (The Lancet, May 20, 2011) describe a method of nerve stimulation by implanting an epidural spinal cord stimulation unit. Upon stimulation, patients were able to stand with balance assistance and eventually voluntarily achieve toe extension, ankle deflection, and leg flexion. The method of Harkema employs an electrode body having slight curvature, which is placed on the dura. The shape and the placement of the electrode body thereby allow a relatively coarse degree of focusing of the electrical current. The device and method of Harkema does not allow for fine control over the location, intensity, phase, and other characteristics of the electrical fields that are applied to the nerve root. Thus, there remains a need for finer control of electrical stimulation.

While these methods have been developed that electrically stimulate the central nervous system or spinal cord, full recovery of movement has not yet been possible. Furthermore, damage to muscles can actually occur with FES when a muscle is contracted by electrical stimulation but opposing muscles are not relaxed as during normal function of a limb, resulting in tears, blisters, or burns. Other problems persons have experienced include dizziness, and autonomic dysreflexia, which is an over-activity of the autonomic nervous system causing an abrupt onset of excessively high blood pressure. Persons can experience discomfort during treatment, such as “pins and needles” under their skin, and a tingling sensation caused by the flow of electrical currents passing through their body. These sensations can be overcome, but the device must be tuned to the user's comfort level (i.e., current type, modulation, waveform, pulse duration and repetition rate, and intensity) or treatment can be unsuccessful. On occasion, the FES electrode's adhesive or gel can cause users to develop skin irritation and rashes. FES treatment is also not recommended for several person groups whose conditions would be sensitive to electrodes.

Therefore, there remains a need for a treatment that addresses neurological damage to the spinal column without adverse affects and that allows a person to regain mobility and a sense of independence, and/or reduce or eliminate various sources of pain.

### SUMMARY OF THE INVENTION

The present invention provides for a motor device for bypassing or bridging an area of neurological damage including at least one electrode including an electric means for generating electric current, and programming means for programming the at least one electrode.

The present invention further provides for a biofeedback system including the motor device in electronic communication with a sensory device including a biofeedback mechanism for sending information generated by the motor device to the spinal cord.

The present invention provides for an artificial spinal cord including the motor device for bypassing an area of neurological damage in communication with a sensory device including a biofeedback mechanism for sending information generated by the motor device to the spinal cord, and the motor device being in communication with an information harvesting device including a mechanism for harvesting information directly from the brain and motor cortex and sending the information to the motor device.

The present invention also provides for a method of stimulating muscle in a person having neurological damage by applying electric current to nerves, bypassing or bridging an area of neurological damage, and causing muscular contraction in a natural manner.

The present invention provides for a method of moving muscles of a paraplegic by applying electric current to nerves, bypassing or bridging an area of neurological damage, and moving normally non-functioning muscles and moving normally non-functioning limbs.

The present invention provides for a method of reducing or eliminating pain from an individual by applying electric current to nerves, bypassing or bridging an area of neurological damage, and reducing or eliminating pain.

The present invention provides for a method of treating foot drop by applying electric current to nerves, bypassing or bridging an area of neurological damage, and regaining feeling and function of a damaged foot.

The present invention further provides for a therapeutic method for exercising for an individual with neurological damage by applying electric current to nerves, bypassing an area of neurological damage, and stimulating and exercising muscles that otherwise would not be stimulated due to the neurological damage.

The present invention provides for a method of generating movement of muscle and sensing that movement in a person having neurological damage by applying electric current to nerves, bypassing an area of neurological damage, moving the muscle in a natural manner, and sending information of the movement to the spinal cord and allowing the person to sense the movement.

The present invention provides for a method of generating movement of muscle using information from the brain and sensing that movement in a person having neurological damage by harvesting information directly from the brain and motor cortex to move muscle, translating the information into the application of electric current to nerves, bypassing an area of neurological damage, moving the muscle in a natural manner, and sending information of the movement to the spinal cord and allowing the person to sense the movement.

The present invention provides for a method of diagnosing neurological damage by applying electric current to nerves, measuring movement of muscle due to the electric current, and based on the amount of muscle movement, diagnosing a person as having neurological damage.

### DESCRIPTION OF THE DRAWINGS

Other advantages of the present invention are readily appreciated as the same becomes better understood by reference to the following detailed description when considered in connection with the accompanying drawings wherein:

FIG. 1 is a top view of the motor device;

FIG. 2 is a view of the motor device in communication with the sensory device;

FIG. 3 is a view of the motor device in communication with the sensory device and the information harvesting device;

FIG. 4 is a view of the motor device that shows one of the available patterns of wire leads and electrode contacts; and

FIG. 5 is a view if the interior of the motor device, showing multiple electrode contacts.

### DETAILED DESCRIPTION OF THE INVENTION

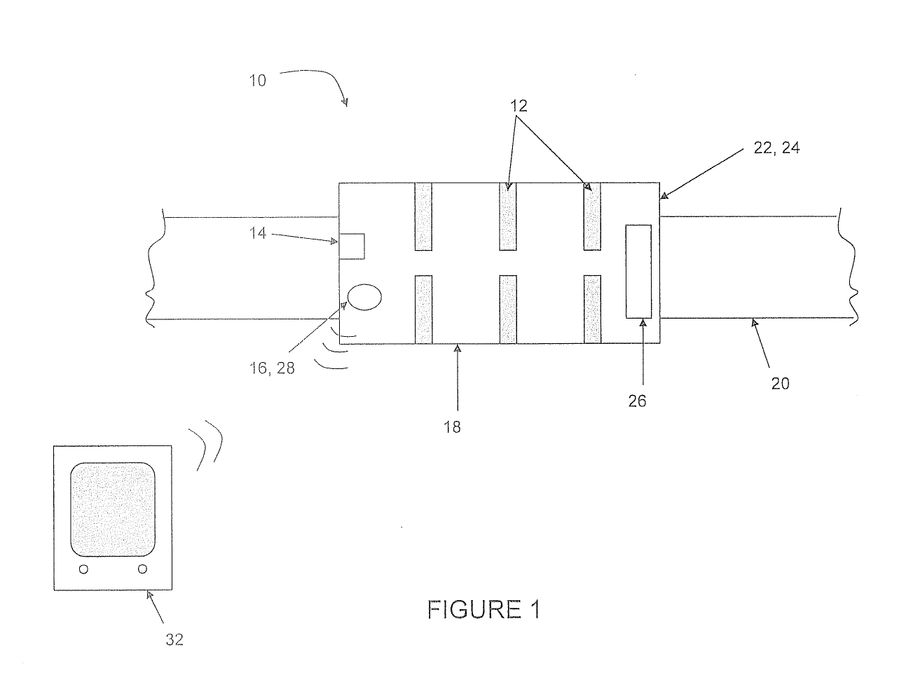

The present invention provides methods of stimulating muscle through electric current that is especially useful in treating paraplegics as well as other kinds of neurological damage. The present invention also provides a motor device, shown generally at 10 in FIG. 1, for bypassing or bridging an area of neurological damage that is used to perform the above method, including at least one electrode 12 having a mechanism 14 for generating electric current, and a programming mechanism 16 for programming the at least one electrode 12. The method and device 10 of the present invention are used to allow an individual with neurological damage to regain use of their muscles and limbs that have been rendered non-functional due to their neurological damage.

The terms “bypass” or “bypassing” as used herein, refer to circumvention of diseased areas of the body, preferably non-functioning neural circuits. Alternatively, this concept can also be referred to as a “bridge” or “bridging” non-functioning neural circuits in the body in order to access functioning neural circuits. These terms can be used interchangeably herein without departing from the spirit of the invention.

The term “neurological damage” refers to any damage relating to nerves or the nervous system. “Neurological damage” can include specifically neural damage wherein neurons are no longer able to communicate and send signals to other neurons in a neural circuit.

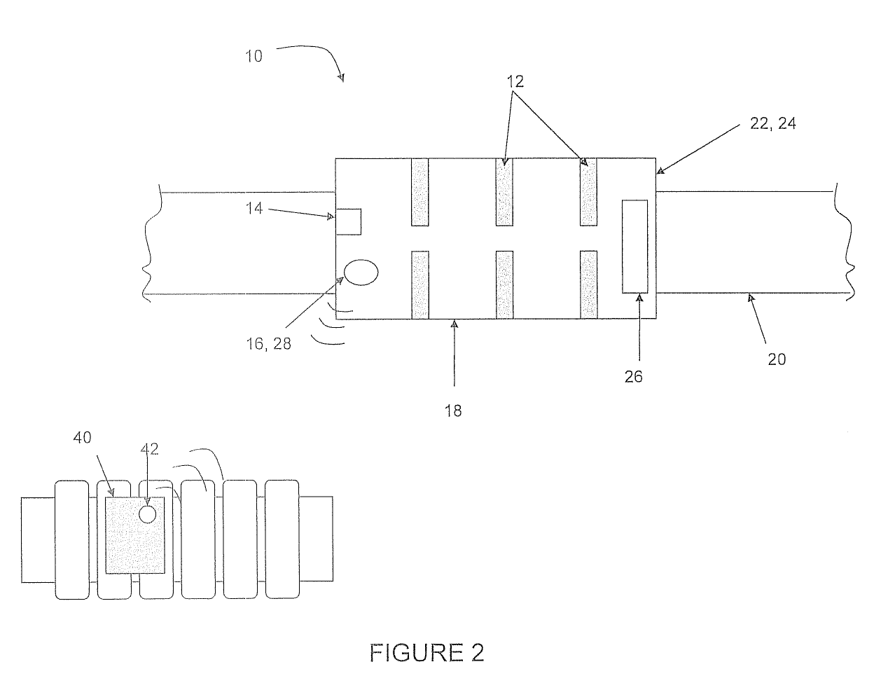

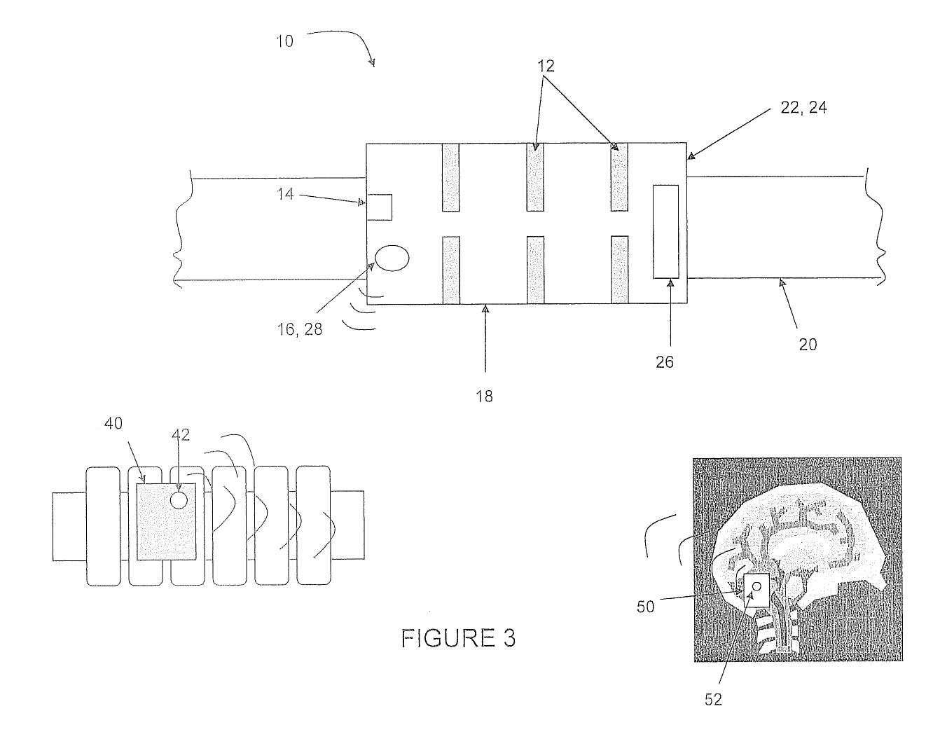

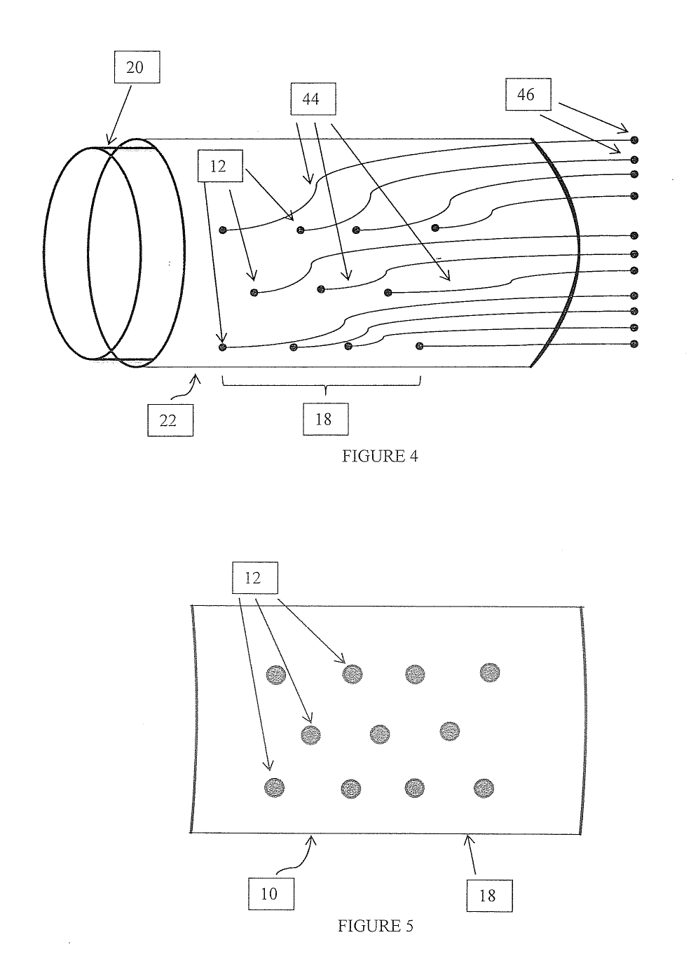

As shown in FIGS. 2 and 3, the motor device 10 can be in the form of a specially designed electrode array 18 that can be deployed around a nerve 20 (preferably the nerve root). The electrode array 18 can be considered as a cuff 22 that hugs the nerve root 20 or a lead 24 that lies or is placed parallel to the nerve root 20 or that can lie or be placed in the epidural space that make contacts 46 with the area requiring electrical stimulation, as shown in FIG. 4. The electrode array 18 can also be positioned in or around any other suitable place for nerve stimulation. Preferably, the electrode array 18 is made of a pliable material, such as, but not limited to, high grade medical polyurethane, polydimethylsiloxane or other silicones, or polyimides such as Pyralin 2611, and is able to easily wrap around the nerve root 20 to conform to its shape.

The electrode array 18 can contain multiple electrodes 12 for independent programming of electrical output, shown for example in FIG. 5. The amount of electrodes 12 used can vary, however, and as many electrodes 12 as possible can be used in the available space in the electrode array 18 in order to stimulate the nerve root 20. The electrode array 18 can be secured into place by tissue glue, sutures, stay screws, or alternatively, the stiffness of the device 10 with specially designed silicon holders with imbedded electrodes 12. With time, scar tissue helps prevent migration of the electrode array 18. By securing the electrode array 18 in a position symmetrical around the nerve root 20, the applied electrical current can be exploited at any particular electrode 12, or combination of electrodes 12, thereby stimulating a more diverse cross-section of nerve roots 20. The electrodes 12 can play off one another in terms of the cathodes and the anodes, as required to avoid tissue damage, and can apply steering current using tripodal electrodes or other arrangements of electrodes, but this allows maximal control in the stimulation of the nerve roots 20 and particularly of nerve fascicles within nerve roots. The principle of motor nerve activation can also be extended to the use of multiple electrode arrays 18, placed on nerves or nerve roots 20 selected for their ability to stimulate muscular contraction. Such multiple electrode arrays may be stimulated in a coordinated fashion, as required, to obtain the desired capabilities that result from muscular contraction, such as locomotion and resolution of foot drop and of arm paresis.

The electrode array 18 can further include a generator or battery 26 that provides electrical current that causes electrical transmission through nerves distal to the point of contact. Preferably, the battery 26 has a long life so that it does not need to be replaced often or require additional surgery. Preferably, the battery 26 includes enough inputs to connect with and to handle various energy output requirements by all of the components of the device 10.

As mentioned above, the electrode array 18 can be programmable and thus includes a programming mechanism 16 such as a computer 28 to execute an algorithm or software and a storage mechanism 30 to store the algorithm or software and any data collected or produced by the algorithm or software. Preferably, the computer 28 includes a user-operated interface 32 that can be programmed or operated by a user, such as a doctor or the person. The computer 28 via the algorithm directly sends signals to the device 10 to cause fluid and natural motion, such as moving a limb or walking. The computer 28 can also communicate wirelessly with a remote interface 32 (shown in FIG. 1), such as, but not limited to, a smart phone, or a touch screen device, a voice-activated device, or a thought-activated device operated directly by the person's own brain signals.

The algorithm can include instructions such as, but not limited to, how often to apply electric current, which nerve to apply current to, and how strong is the applied current. Additional parameters can be programmed and can include any combination of the following: the timing of electrical potential applied at different electrodes 12 in the electrode array 18 and/or in multiple electrode arrays used on a multiplicity of nerves and/or nerve roots 20; varying the intensity of electrical current applied at different electrodes 12 in the electrode array 18; the use of variable frequency trains; relaxation kinetics; stimulation frequency; shortening history; and random modulation of parameters, including: constant stimulation, randomized frequency, randomized current amplitude, and randomized pulse width. The battery 26, leads 24, electrodes 12, and program parameters are all adjusted to minimize pain felt by the person.

Most generally, a method of stimulating muscle in a person having neurological damage is provided by applying electric current to nerves, bypassing or bridging an area of neurological damage, and moving the muscle in a natural manner. Preferably, this method is performed with the motor device 10 as described above.

The electric current can be applied to various nerves, including, but not limited to, central nerves, nerve roots, or peripheral nerves. Nerve roots carry very specific information to well mapped out myotomes and therefore this information can be exploited in this mapping to stimulate the muscle groups required to cause motion. Preferably, electric current is applied to a nerve bundle in order to stimulate a muscle group instead of just a single muscle, in order to provide natural movement. In other words, the stimulation is a coordinated muscle group stimulation. Electric current can be applied by inserting the motor device 10 including electrodes 12 into the spinal canal, or selected peripheral nerves, so that they become in intimate contact with nerve roots therein. Alternatively, the device 10 including electrodes 12 can be placed in close proximity of specific sensory (afferent) neurons. As described above, the electrode array 18 of the device 10 can be wrapped around the nerve root 20 or lie parallel to the nerve root 20.

More specifically, the motor device can be inserted by a method such as, but not limited to, translaminar percutaneous insertion, translaminar insertion via surgical laminotomy, surgical foraminotomy, and surgical implantation around and adjacent to a peripheral nerve. The extraforaminal route places the device 10 directly over the nerve root. The translaminar route places the device 10 around the nerve roots. With an intradural route, the dura is opened and the device 10 is placed directly over a nerve root, resulting in a very sensitive placement. Epidural stimulation adjacent to the nerve root can also stimulate the nerve root, and epidural placement in the thoracic spine or cervical spine area can be used to stimulate the spinal cord and the variety of movements can be specifically programmed. The use of multiple devices 10 moreover can be used for coordinated stimulation of major muscle groups used for walking and other activities.

As a result of the electric potential provided by the electrodes 12, nerve signals can cross the neuromuscular synapse and bypass or bridge areas of neurological damage, thereby causing muscles to contract or relax as needed. More specifically, in the preferred embodiment of the invention, the electric current is applied to an area right above an area of neurological damage, where it bypasses or bridges the area of neurological damage, and travels to an area below the damage in order to stimulate muscle. This process requires an understanding of where a neurological signal was coming from above the area of neurological damage as well as where that signal needs to travel to, such as directly in the spinal cord or in a muscle or muscle group itself, in order to correctly stimulate muscle. This is an important process in the present invention. One of the main reasons that people with neurological damage cannot function as normal and move their limbs is because the muscles required for movement cannot receive signals from neurons to stimulate the muscles. There is generally an area along the synaptic pathway that is damaged such that a signal generated in the brain to move the muscle cannot reach its intended target muscle due to this damage. By creating a bypass, electric current as applied in the present invention can reach the intended target muscle, allowing an individual to move that muscle as normal. Thus the present invention can precisely target motor nerve roots, and also peripheral neurons that are responsible for general or specific areas of the body and that can be injured such that their function is compromised, essentially reversing the effects of neurological damage.

Not only can the electric current can be applied above an area of neurological damage and bypass or bridge damaged nerve areas as stated above, but also below an area of neurological damage directly without the need for a bypass or bridge. Therefore, the present invention provides for a method of stimulating muscle in a person having neurological damage by applying electric current to nerves, thereby moving the muscle in a natural manner. This method is especially useful when a very specific muscle group is desired to be moved or a person only desires to have their muscle perform a specific function. In this case, it is not necessary to bypass or bridge an area of neurological damage.

The methods of the present invention, along with the appropriate contacting electrodes 12, and computer algorithm(s) of the motor device 10, are useful to allow person control, and are intended to allow persons to move digits, limbs and other body parts that have become paralyzed due to trauma and/or disease resulting from a broad spectrum of causes and especially by loss of nerve function. Thus, in the successful use of this invention, a paralyzed individual can become capable of standing, flexing muscles, and motility such as walking. In other words, use of the device 10 of the present invention can allow an otherwise paralyzed person to regain function of their body. This invention can be used as an interventional treatment for persons who are paralyzed due to spinal cord involvement. This invention can be used as an interventional treatment for persons having arm paresis due to a cerebrovascular accident or stroke. This invention can also be used to treat persons who, due to spinal cord injury, have lost feeling in specific parts of the body. Additionally, this invention can be used to reduce or eliminate the feeling of pain felt at peripheral locations by those individuals who feel chronic pain. The methods and device 10 disclosed herein can be used with any individual that has neurological damage.

The present invention provides more specifically for a method of moving muscles of a paraplegic, by applying electric current to nerves, bypassing or bridging an area of neurological damage, and moving normally non-functioning muscles, and thereby moving normally non-functioning limbs. By using the motor device 10 as explained above, this method can allow a paraplegic to regain function of any part of the body that had been rendered non-functioning by their condition. For example, by applying electric current to appropriate nerves, muscles required for walking can be stimulated and moved, thereby allowing the individual to walk by moving their legs.

The present invention further provides for a method of reducing or eliminating pain from an individual, by applying electric current to nerves, bypassing or bridging an area of neurological damage, and reducing or eliminating pain. Preferably, this method is performed by using the device 10 described above. The electric current acts to influence the processing of information within the central nervous system, and increase peripheral blood flow. The intrinsic nervous system of a muscle is interposed between the information processing of the central nervous system and muscle function, so the electric current can modulate the processing of the pain experienced at the muscle.

The present invention further provides for a method of treating foot drop, by applying electric current to nerves, bypassing or bridging an area of neurological damage, and regaining feeling and function of a damaged foot. In foot drop, the individual is unable to lift, or finds difficulty in lifting, the front of the foot when walking. This method is preferably performed by using the device 10 as described above.

This condition can result from direct injury to the spinal cord, along with degenerative conditions such as multiple sclerosis. A recent treatment for this condition uses the Ness L300 system (Bioness, Inc, Valencia, Calif.). This system provides electrical stimulation to peripheral muscles, specifically the anterior tibialis, in response to the foot being lifted from the ground. The present invention provides a distinct improvement on the Ness L300. In the resulting improvement, the electrical stimulation is directed at the nerve root in the spinal cord, for example, at neurologic level L4, thereby allowing the individual to both flex the foot and lift the leg while walking, which more closely resembles the gait of a healthy individual. Along with allowing greatly improved motility, the use of the present invention can reverse or dramatically diminish the extent muscle atrophy.

The method of the present invention can be used as a combination treatment with other therapies that are currently used for treating neurological damage, or whose use is currently under investigation. For example, the present invention provides for a method of stimulating muscle in a person having neurological damage by treating the person with stem cells, applying electric current to nerves, bypassing or bridging an area of neurological damage, and moving the muscle in a natural manner. This method is especially helpful when the current therapy can be expected take months or years to take effect, if at all. Stem cell therapy applied to the spinal column may not produce results for five years or more. In the meantime, by combining the stem cell treatment with the present invention, muscle can be stimulated, exercised, and strengthened in anticipation of the treatment being effective. Thus, the electrical stimulation can have an adjuvant effect with stem cell treatment in providing the restoration of natural function. This method is preferably performed by using the device 10 as described above.

The method of the present invention can also be used as a therapeutic method for exercising for an individual with neurological damage by applying electric current to nerves, bypassing an area of neurological damage, and stimulating and exercising muscles that otherwise would not be stimulated due to the neurological damage. This method allows an individual's muscles to be exercised through contraction and relaxation and to grow stronger over time due to the stimulation by electric current. This method of exercise is more natural than by exercising with machines or a physical therapist, as the there is less risk of damage to muscles due to natural movement through the electrical stimulation. While an individual may not be strong enough at first to use certain muscles, over time by performing this method, the individual can build stronger muscles and eventually use the limbs that the muscles control. This method is preferably performed by using the device 10 as described above.

With all of the methods of the present invention, therapy and rehabilitation with a therapist can be required to train a person to become comfortable with the capabilities of the device as well as the restrictions of their own body. Initially, the movements can be difficult to control, and working with a therapist allows for the right parameters to be programmed into the device 10 to provide natural movement. This also allows the person to get used to the flow of current and the meaning of the current in terms of movement of the body.

The device 10 and methods of the present invention can also be used in combination with a mobile standing device, such as, but not limited to, the STANDING DANI® (Davis Made, Inc.) as well as other such assistive devices. Once the device 10 is implanted in the person, the mobile standing device can be a failsafe where the person can gradually become further and further in control of their own legs as they become comfortable with the programming and become stronger. The device 10 can be used in like fashion in combination with other such assistive devices, another example of which includes devices for upper limb rehabilitation used to regain arm function following stroke. Thus, device 10 can broadly be applied in cases of paralysis or other movement-related disorders of the body.

The device 10 of the present invention overcomes the problems of electrical stimulation devices of the prior art because multiple muscles or areas of muscles can be stimulated at once, thereby allowing for natural movement of muscles and elimination of damage of muscles due to contraction without corresponding relaxation. An indirect positive outcome is that causing muscular contraction can stimulate bone regeneration, thereby making bones stronger, or at least reducing the rate of bone loss. The use of motor nerve roots is a more elegant solution than is the use of peripheral transcutaneous stimulation or transepithelial stimulation (TES): it is applied at the spinal cord at the level of injury, can eliminate the muscle tears, blisters, rashes, burns, and dizziness found with percutaneous TES. This method can overcome a major disadvantage of poor stimulation selectivity and allow more natural walking patterns than surface electrodes, thus being more suited as a prosthetic device for chronic use.

The motor device 10 can also be electronically connected to a sensory device 40, shown in FIG. 2 (not shown to scale), having a biofeedback mechanism 42 to send information to the spinal cord and the brain as a part of a biofeedback loop so that the person can “sense” the movement. Information harvested from the motor stimulation can be sent to a spot above the level of spinal cord injury to an intact segment to then help the body “feel” the movement. The information can be sent by wired communication or wireless communication between the motor device 10 and the sensory device 40. The sensory device 40 can further include computer storage and algorithm mechanisms to control and send information. Both the motor device 10 and the sensory device 40 can be independently programmable via an external source. Therefore, the present invention provides for a biofeedback system including the motor device 10 in electronic communication with the sensory device 40 having the biofeedback mechanism 42 to send information generated by the motor device 10 to the spinal cord.

The present invention further provides for a method of generating movement of muscle and sensing that movement in a person having neurological damage, by applying electric current to nerves, bypassing an area of neurological damage, moving the muscle in a natural manner, and sending information of the movement to the spinal cord, thereby allowing the person to sense the movement. Preferably, this method is performed by using the motor device 10 and sensory device 40 as described above. Each of these steps has been described above, with the applying, bypassing, and moving steps being performed by the motor device 10, and the sending (and sensing) step being performed by the sensing device 40. This method allows an individual with neurological damage and inability to move muscles in a normal manner to move those muscles as well as sense that movement.

In one example of this method, if a person has a T10 paraplegia, the device 10 can be hooked up such that it now bridges the T10 injury at approximately the T8 or T7 level and as the device 10 is turned on, unique sensory signals can be sent back to the brain via the intact spinal cord at around the T7 level, thereby creating a biofeedback loop and the person is then aware of the motion of their legs through true sensory patterns which are unique for every particular motion.

Also, the biofeedback loop can be completed by linking the motor device 10 to an information harvesting device 50 that includes a mechanism 52 for harvesting information directly from the brain and motor cortex, as shown in FIG. 3. The information harvesting device 50 can then send information to the motor device 10 to create a complete parallel structure referred to as an “artificial spinal cord”. Again, the information can be sent by wired communication or wireless communication between the information harvesting device 50 and the motor device 10. This allows the person to directly control the movement of muscles that are stimulated by the motor device 10 instead of relying on a program to activate the electrical stimulation of the muscles. The information harvesting device 50 can further include any computer or algorithm mechanisms as necessary to communicate with the motor device 10. Essentially, the information transmitted by the information harvesting device 50 is translated by the motor device into electric current needed to apply to the nerves.

Therefore, the present invention provides for an artificial spinal cord including the motor device 10 as described above for bypassing an area of neurological damage in communication with the sensory device 40 having the biofeedback mechanism 42 to send information generated by the motor device 10 to the spinal cord, and the motor device 10 being in communication with the information harvesting device 50 having the mechanism 52 to harvest information directly from the brain and motor cortex and send to the motor device 10.

The present invention further provides for a method of generating movement of muscle and sensing that movement in a person having neurological damage, by harvesting information directly from the brain and motor cortex to move muscle, translating the information into the application of electric current to nerves, bypassing an area of neurological damage, moving the muscle in a natural manner, and sending information of the movement to the spinal cord, thereby allowing the person to sense the movement. Each of these steps is described above and these are preferably performed by the motor device 10, the sensory device 40, and the information harvesting device 50, and by performing this method, a person having neurological damage can bypass their own spinal cord and areas of neurological damage. The person can generate the signal to move their own muscles, allowing them to move in a natural manner. This method is especially useful to those people who have significant damage to their own spinal cord.

The motor device 10 of the present invention can further be used in a diagnostic method by applying electric current to nerves, measuring movement of muscle due to the electric current, and based on the amount of muscle movement, diagnosing a person as having neurological damage. This method can be used to determine whether there has been neurological damage to an area of the body, as well as to determine whether a certain amount of electric current can bypass the area of neurological damage to stimulate and move the muscle. If a muscle fails to move or moves less than expected, then neurological damage has occurred. This method can be used to determine how effective the electric current is at bypassing the area of neurological damage and moving the muscle. The steps of this method are essentially performed as describe above. The electric current can be applied to any nerves in the body in this method.

The present invention provides many advantages over the prior art. The present invention utilizes circumferential electrode bodies that maintain direct contact around the circumference of the nerve root. A multiplicity of electrical contacts are placed within the electrode body and thereby are placed in immediate proximity to the nerve. These multiple contacts are placed in direct contact with the nerve surface, so to allow a fine degree of control over the location, intensity, phase and other characteristics of each of the electrical fields that are applied to the nerve root. This is of particular significance when stimulating a nerve root, due to the presence of multiple nerve fascicles within the nerve root. Thus, the present method allows a very fine degree of control in stimulating nerves within the nerve roots, which by its design improves on prior art methods described above. Direct circumferential contact of nerves, by the device in the present invention, thus has advantages that include a finer and more targeted current applied directly to the surface of the nerve; reduced current demands that lower the potential for tissue damage and minimize energy consumption; and more consistent contact of all electrical contacts within the electrode body.

The invention is further described in detail by reference to the following experimental examples. These examples are provided for the purpose of illustration only, and are not intended to be limiting unless otherwise specified. Thus, the present invention should in no way be construed as being limited to the following examples, but rather, be construed to encompass any and all variations which become evident as a result of the teaching provided herein.

### Example 1

Under the supervision of the onsite veterinarian, and under aseptic conditions, rats were anesthetized and kept under conscious sedation with appropriate analgesics, as per the veterinarian. Local anesthetics were injected and a midline incision was made getting down to the lamina. Laminontomies and laminectomies were performed utilizing a high speed Midas Rex Drill. The dura and nerve roots were exposed and epidural leads were directly placed over the top of the nerves. Electrodes were subsequently connected to the neuromodulator device, which when activated caused motor contractions in the corresponding muscles. Quantitative results could not be obtained due to the large size of the electrodes relative to the very small fine nerves of the rat, which caused difficulty in reproducible electrode placement. However, the observation was made in every case that spinal cord nerve root stimulation caused motor contractions in the corresponding muscles.

### Example 2

In the course of electrode placement for treatment of pain in a paralyzed person, an eight-contact epidural lead was placed at the foramen covering the nerve roots at neurological levels L5 and S1. This procedure was performed by a neurosurgeon using a cannulated catheter and was guided by fluoroscopy. The act of programming of the spinal cord stimulator caused the person's foot to flex. This result indicates that motor nerve root stimulation in the spinal cord can, indeed, cause contraction of corresponding muscles.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A motor device for bypassing or bridging an area of neurological damage, comprising:
at least one electrode including electric means for generating electric current, and programming means for programming the at least one electrode.

2. The motor device of claim 1, wherein said motor device is in the form of an electrode array including at said least one electrode.

3. The motor device of claim 2, wherein said electrode array is chosen from the group consisting of a cuff and a lead.

4. The motor device of claim 2, wherein said electrode array is made of a pliable material.

5. The motor device of claim 2, further including securing means for securing said motor device in a body chosen from the group consisting of tissue glue, sutures, and stay screws.

6. The motor device of claim 5, further including a battery.

7. The motor device of claim 6, wherein said programming means is further defined as a computer and further including an information storage mechanism.

8. The motor device of claim 7, wherein said computer includes a user-operated interface.

9. The motor device of claim 8, wherein said computer is in electronic communication with a remote interface chosen from the group consisting of a smart phone, a touch screen device, a voice-activated device, and a thought-activated device.

10. The motor device of claim 7, wherein said computer includes means for varying parameters chosen from the group consisting of timing of electrical potential applied at different electrodes in said electrode array, varying the intensity of electrical current applied at different electrodes in said electrode array, use of variable frequency trains, relaxation kinetics, stimulation frequency, shortening history, constant stimulation, randomized frequency, randomized current amplitude, and randomized pulse width.

11. A biofeedback system comprising the motor device of claim 1 in electronic communication with a sensory device including biofeedback means for sending information generated by said motor device to the spinal cord.

12. The biofeedback system of claim 11, wherein said sensory device is programmable and includes computer storage and algorithm means for controlling and sending information.

13. An artificial spinal cord, comprising:
the motor device of claim 1 for bypassing an area of neurological damage in communication with a sensory device including biofeedback means for sending information generated by said motor device to the spinal cord, and said motor device being in communication with an information harvesting device including means for harvesting information directly from the brain and motor cortex and sending said information to said motor device.

14. A method of stimulating muscle in a person having neurological damage, including the steps of:
applying electric current to nerves;
bypassing or bridging an area of neurological damage; and
moving the muscle in a natural manner.

15. The method of claim 14, wherein the nerves are chosen from the group consisting of central nerves, nerve roots, and peripheral nerves.

16. The method of claim 14, wherein said applying step is further defined as applying electric current to a nerve bundle and stimulating a muscle group.

17. The method of claim 14, wherein said applying step further includes the step of inserting a motor device including electrodes in a person in proximity to the nerves.

18. The method of claim 17, wherein said inserting step further includes a step chosen from the group consisting of wrapping an electrode array around a nerve root, placing the electrode array parallel to a nerve root, and placing the electrode array in epidural space.

19. The method of claim 18, wherein said inserting step is performed by a method chosen from the group consisting of translaminar percutaneous insertion, translaminar insertion via surgical laminotomy, surgical foraminotomy, and surgical implantation around and adjacent to a peripheral nerve.

20. The method of claim 14, wherein said applying step is further defined as applying electric current to an area above the area of neurological damage and said bypassing step causes the electric current to travel to an area below the area of neurological damage.

21. The method of claim 14, wherein said moving step is further defined as moving a part of the body chosen from the group consisting of at least one digit and at least one limb.

22. The method of claim 14, wherein said moving step is further defined as an action chosen from the group consisting of flexing muscles, standing, walking, and combinations thereof.

23. The method of claim 14, wherein said moving step is further defined as strengthening muscles.

24. The method of claim 14, further including the step of treating the person with stem cells prior to said applying step.

25. The method of claim 14, wherein said moving step further includes the step of using a device chosen from the group consisting of a mobile standing device, an assistive device, and a rehabilitative device.

26. A method of moving muscles of a paraplegic or a person who suffers from other movement-related disorders of the body, including the steps of:
applying electric current to nerves;
bypassing or bridging an area of neurological damage; and
moving normally non-functioning muscles and moving normally non-functioning limbs.

27. The method of claim 26, wherein said applying step is performed with the motor device of claim 1.

28. The method of claim 26, wherein said moving step is further defined as moving muscles required for walking, and moving the legs of the paraplegic to walk.

29. The method of claim 26, wherein said moving step is further defined as moving muscles required for use of the arms and hands.

30. A method of reducing or eliminating pain from an individual, including the steps of:
applying electric current to nerves;
bypassing or bridging an area of neurological damage; and
reducing or eliminating pain.

31. The method of claim 30, wherein said applying step is performed with the motor device of claim 1.

32. The method of claim 31, wherein said reducing or eliminating pain step is further defined as influencing the processing of information within the central nervous system and increasing peripheral blood flow.

33. A method of treating foot drop, including the steps of:
applying electric current to nerves;
bypassing or bridging an area of neurological damage; and
regaining feeling and function of a damaged foot.

34. The method of claim 33, wherein said applying step is performed with the motor device of claim 1.

35. A therapeutic method for exercising for an individual with neurological damage, including the steps of
applying electric current to nerves;
bypassing an area of neurological damage; and
stimulating and exercising muscles that otherwise would not be stimulated due to the neurological damage.

36. The method of claim 35, wherein said applying step is performed with the motor device of claim 1.

37. The method of claim 36, wherein said stimulating and exercising step is further defined as contracting and relaxing muscles.

38. A method of generating movement of muscle and sensing that movement in a person having neurological damage, including the steps of:
applying electric current to nerves;
bypassing an area of neurological damage;
moving the muscle in a natural manner; and
sending information of the movement to the spinal cord and allowing the person to sense the movement.

39. The method of claim 38, wherein said applying step is performed by the motor device of claim 1 and said sending step is performed by a sensory device including biofeedback means for sending information generated by the motor device to the spinal cord.

40. A method of generating movement of muscle using information from the brain and sensing that movement in a person having neurological damage, including the steps of:
harvesting information directly from the brain and motor cortex to move muscle;
translating the information into the application of electric current to nerves;
bypassing an area of neurological damage;
moving the muscle in a natural manner; and
sending information of the movement to the spinal cord and allowing the person to sense the movement.

41. The method of claim 40, wherein said method is performed by the artificial spinal cord of claim 13.

42. A method of diagnosing neurological damage, including the steps of:
applying electric current to nerves;
measuring movement of muscle due to the electric current; and
based on the amount of muscle movement, diagnosing a person as having neurological damage.

43. The method of claim 42, wherein said applying step is performed by the motor device of claim 1.

44. The method of claim 43, wherein said measuring step further includes the step of determining whether an amount of electric current can bypass the area of neurological damage to stimulate and move the muscle.
